(19) United States
(12) Patent Application Publication
(10) Pub. No.: US 2011/0295195 A1
(43) Pub. Date: Dec. 1, 2011

(54) OPHTHALMIC SURGICAL CASSETTES WITH IDENTIFICATION FEATURES

(76) Inventors: James T. Perkins, St. Charles, MO (US); David W. Hertweck, Valley Park, MO (US)

(21) Appl. No.: 13/091,279

(22) Filed: Apr. 21, 2011

Related U.S. Application Data
(60) Provisional application No. 61/348,407, filed on May 26, 2010.

(51) Int. Cl. A61M 1/00

(52) U.S. Cl. 604/35

(57) ABSTRACT

An ophthalmic surgical cassette for use with an ophthalmic surgical console. The ophthalmic surgical cassette includes a rigid-walled container having an interior volume for collecting aspirant fluid and/or tissue, and a manifold base, including an irrigation tube for coupling an irrigation source, an aspiration tube for coupling an ophthalmic surgical handpiece, and an identification feature for indicating a cassette type to an ophthalmic surgical console. The manifold base is removeably attached to the rigid-walled container.

### CROSS-REFERENCE TO RELATED APPLICATIONS

This application claims the benefit of U.S. Provisional Application No. 61/348,407, filed May 26, 2010, the entire disclosure of which is incorporated herein by reference.

### BACKGROUND

1. Field

The present invention is directed to ophthalmic surgical cassettes for use with ophthalmic surgical consoles. More specifically, the present disclosure is directed towards an ophthalmic surgical cassette having a removable manifold base, including an identification feature for indicating a cassette type to an ophthalmic surgical console.

2. Description of the Related Art

The statements in this section merely provide background information related to the present disclosure and may not constitute prior art.

Ophthalmic surgical cassettes for use with pump systems during ophthalmic surgical procedures are generally known. Ophthalmic surgical cassettes commonly include a container for retaining aspirant fluid and tissue retrieved from a surgical site during an ophthalmic surgical procedure. It is known for ophthalmic surgical cassettes to also include a fluid level indicator, which indicates to a surgeon when an ophthalmic surgical cassette has retained a pre-determined volume of aspirant fluid and tissue. Further, a variety of identification schemes are similarly known in the art to signal to a type of ophthalmic surgical cassette.

There exists the need for an ophthalmic surgical cassette with an improved identification feature for indicating a cassette type to an ophthalmic surgical console.

### BRIEF DESCRIPTION OF THE DRAWINGS

The drawings described herein are for illustration purposes only and are not intended to limit the scope of the present disclosure in any way.

FIG. 1 is a perspective view of an ophthalmic surgical cassette, according to the present disclosure;

FIG. 2 is a perspective view of an ophthalmic surgery pump system, including the ophthalmic surgical cassette of FIG. 1, according to the present disclosure;

FIG. 3 is a partial perspective view of the identification feature of the ophthalmic surgical cassette of FIG. 1;

FIG. 4 is a graphical illustration of the refraction of light by the identification feature of the ophthalmic surgical cassette of FIG. 1;

FIG. 5 is an illustration of the light waveform detected from the ophthalmic surgical cassette of FIG. 1;

FIG. 6 is a partial perspective view of an identification feature, according to one embodiment of the present disclosure;

FIG. 7 is an illustration of the light waveform detected from an ophthalmic surgical cassette, including at least one treated surface; and

FIG. 8 is a perspective view of a manifold base, including an aspiration tube and an auxiliary aspiration tube, according to the present disclosure.

### DETAILED DESCRIPTION OF THE PREFERRED EMBODIMENT

The following description is merely exemplary in nature and is not intended to limit the present disclosure, application, or uses.

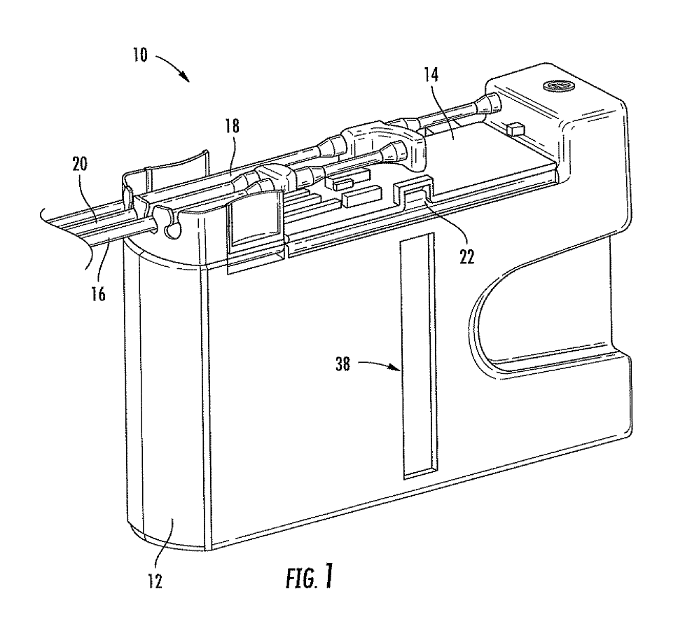

According to one embodiment of the present disclosure, an ophthalmic surgical cassette 10 is illustrated in FIG. 1. The ophthalmic surgical cassette 10, includes a rigid-walled container 12, having an interior volume for collecting aspirant fluid and/or tissue, and a manifold base 14, including an irrigation tube 16 for coupling an irrigation source (not shown), and an aspiration tube 18 for coupling an ophthalmic surgical handpiece (not shown). The manifold base 14 is removeably attached to the rigid-walled container 12, such that the ophthalmic surgical cassette 10 may be emptied during an ophthalmic surgery procedure, without disconnecting the irrigation tube 16 from the irrigation source or the aspiration tube 18 from the ophthalmic surgical handpiece. The manifold base 14, further includes an irrigation tube 20 for providing irrigation to a surgical site.

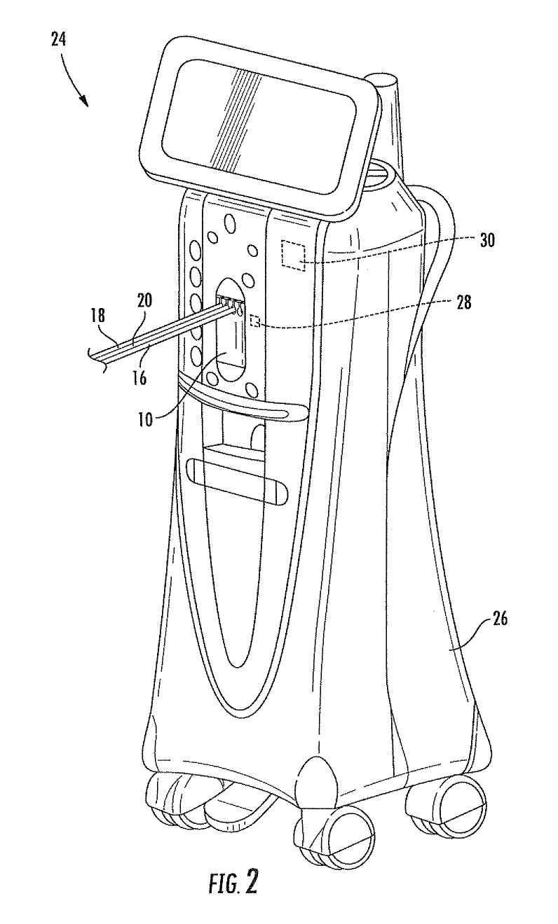

The manifold base 14 also includes an identification feature 22 for indicating a cassette type to an ophthalmic surgical console. In this embodiment, the cassette type indicated by the identification feature 22 is anterior. An example embodiment of an ophthalmic surgical console is illustrated in FIG. 2 and described below. During an ophthalmic surgical procedure (except when being emptied), the ophthalmic surgical cassette 10 is disposed at least partially within an ophthalmic surgical console. The cassette type indicates to the ophthalmic surgical console which of one or more ophthalmic surgical procedures the ophthalmic surgical cassette 10 is configured to perform. Each ophthalmic surgical procedure may include multiple surgical functions, which the ophthalmic surgical console enables upon detecting the identification feature. In this manner, only the surgical functions usable by a particular cassette type are enabled for use by a surgeon, while other surgical functions may be disabled. Surgical functions may be enabled/disabled individually or associated with a surgical routine, such that all surgical functions in the surgical routine are enabled/disabled together, or some combination thereof. For example, when a cassette type is anterior, as in FIG. 1, an ophthalmic surgical console may disable surgical routines for posterior and/or combined (anterior and posterior) ophthalmic surgical procedures.

In one example, when a posterior cassette type is indicated by the identification feature of an ophthalmic surgery cassette, a surgical function potentially unused during a posterior procedure, e.g., phacoemulsification, etc., may be disabled by the ophthalmic surgical console. In at least one embodiment of the present disclosure, a user may be forced to remove an incorrect ophthalmic surgical cassette and install a correct ophthalmic surgical cassette before an intended ophthalmic surgical procedure may be performed. Thus, an identification feature according to the present disclosure may ensure use of an ophthalmic surgical cassette in an appropriate ophthalmic surgical procedure, providing a potential safety feature.

In various embodiments of the present disclosure, the types and arrangements of tubes on a manifold base of an ophthalmic surgical cassette, in addition to other components, e.g., a reflux bulb, etc., often dictate which types of ophthalmic surgical procedures may be completed using said ophthalmic surgical cassette. Accordingly, an identification feature included in the manifold base, as compared to other components of an ophthalmic surgery cassette, provides a more accurate indication of the one or more surgical procedures for which the ophthalmic surgical cassette is intended. Further, when a manifold base is removable, e.g., removable manifold base 14 illustrated in FIG. 1, etc., the manifold base may be configured to receive a generic or universal size of container for convenience in manufacturing. If an indication scheme were implemented on a generic container or a container with a generic interface to a manifold base, containers may be mixed up and coupled to a manifold base configured for different types of ophthalmic surgical procedures, thereby providing an errant indication to an ophthalmic surgical console. As a result, the ophthalmic surgical console may disable surgical functions useable with the ophthalmic surgical cassette and/or the manifold base, and enable surgical functions not useable with the ophthalmic surgical cassette and/or the manifold base. Remedying the errant indication generally requires identification of the ophthalmic surgical cassettes, including swapped container (not always an easily visible indicator), and un-swapping the container. According to the present disclose, an identification feature included in a manifold base of an ophthalmic surgical cassette eliminates at least this potential error, even when a generic rigid-walled container is included.

FIG. 2 illustrates an embodiment of an ophthalmic surgery pump system 24, according to the present disclosure. The ophthalmic surgery pump system 24 is an ophthalmic surgical system for anterior, posterior, or combined procedures, including an ophthalmic surgical console 26 and the ophthalmic surgical cassette 10 installed in the ophthalmic surgical console 26.

The ophthalmic surgical console 26 includes a detector 28 disposed internal to the ophthalmic surgical console 26 (shown in broken lines) and adjacent to the ophthalmic surgical cassette 10 for detecting the identification feature 22, In this particular embodiment, the detector 28 includes a light source and a linear array of charged coupled devices (CCD). In other embodiments, a different arrangement of CCDs may be employed depending on a configuration of an ophthalmic surgical cassette and/or an ophthalmic surgical console. In still other embodiments, a detector may include a photo-detector, such as a CMOS device, a contact image sensor, or a different suitable device, etc., for detecting various types of identification features, through light transmission and/or otherwise. A detector may be disposed in one or more different locations, e.g., internal and/or external to an ophthalmic surgical console, etc., as long as the detector is disposed for detecting an identification feature included in a manifold base of an ophthalmic surgical cassette.

The detector 28 is operably coupled to a processor 30 (shown in broken lines) disposed internal to the ophthalmic surgical console 26. The processor 30 is operably coupled to the detector 28, such that when the detector 28 detects an identification feature, the processor 30 receives an input from the detector 28, and determines a cassette type of an ophthalmic surgical cassette installed in the ophthalmic surgery pump system 24. Then, the processor 30 may enable and/or disable surgical functions/routines appropriate to the installed ophthalmic surgical cassette. It should be appreciated that a processor may include a number of different processing devices, as well known in the art. For example, a processor may include a microprocessor, CPU, microcontroller, gate array, logic circuit, or one or more other suitable devices, etc.

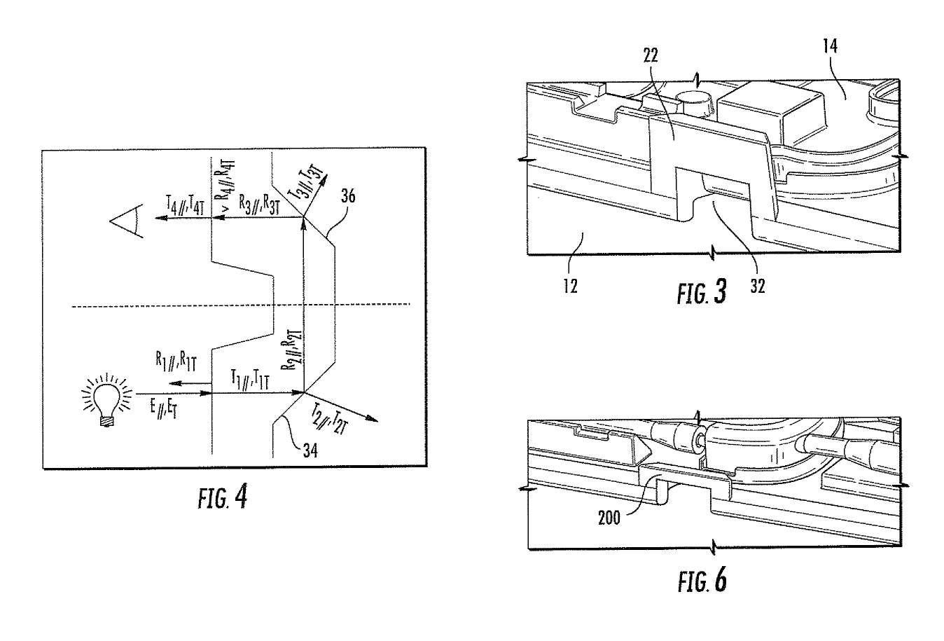

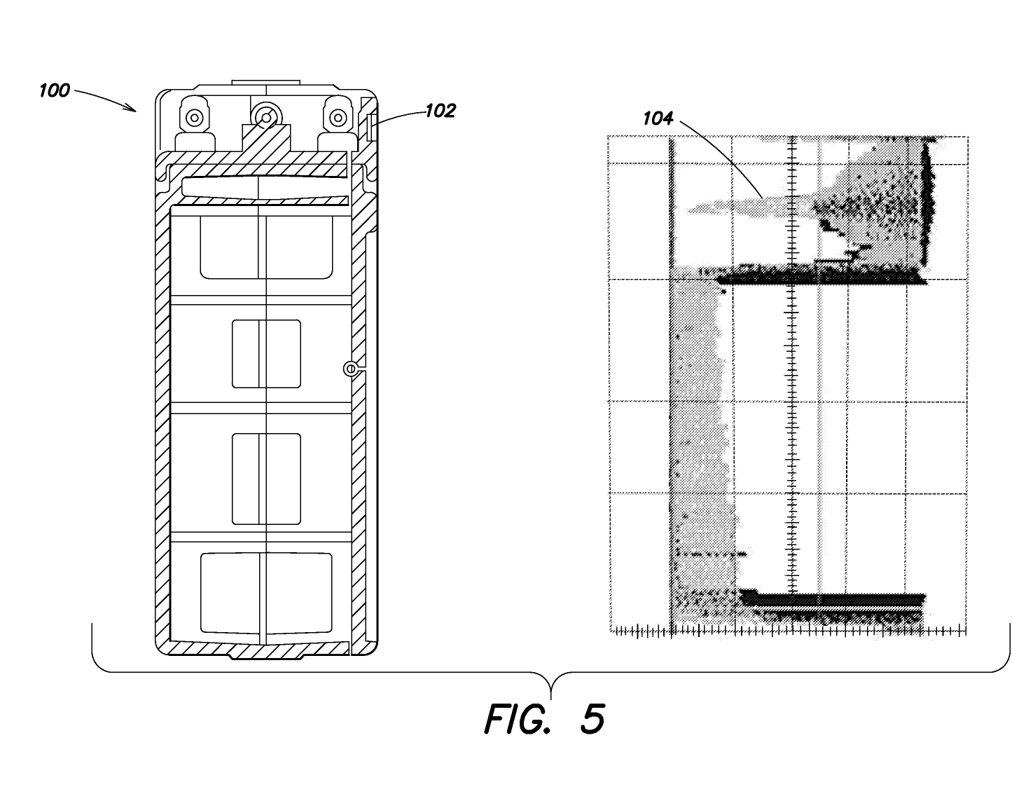

As shown in the detailed view of FIG. 3, the identification feature 22 includes a refraction element for refracting light transmission generated from the ophthalmic surgical console 26. The refraction element includes a notch section 32, forming a prism. Light transmission relative to the identification feature 22 is illustrated in the diagram of FIG. 4, not drawn to scale relative to FIG. 3. Light emitted from a light source (denoted by the light bulb symbol) reflects from angled portion 34 (a boundary between two mediums), to angled portion 36 (a boundary between two mediums), and to the detector 28 (denoted by the eye symbol). The light transmission is reflected very efficiently through the identification feature 22 and causes a spike of intensity in detector 28. Specifically as illustrated in FIG. 5, a spike 104 is detected, which corresponds to light refracted from refraction element 102 of an anterior ophthalmic surgical cassette 100. The processor 30 calculates the slope of the spike 104, and based on the position of the slope (defined by the detector 28, e.g., linear CCD array, etc.), the cassette type may be determined by the processor 30.

Various aspects of a refraction element, e.g., type of material, the angle of sides, dimensions of sides walls/faces, etc., may be altered in other embodiments to change one of more characteristics of the light refracted, e.g., wavelength, speed, slope, position, intensity, frequency, etc., to indicate a number of distinct cassette types. For example as shown in FIG. 6, an identification feature 200 includes different dimensions, as compared to the identification feature 22, to indicate a cassette type different than the cassette type of FIG. 1. Accordingly, because the aspects of a refraction element may be varied to provide any number of unique spikes, numerous different cassette types may be indicated by an identification feature, including a refraction element.

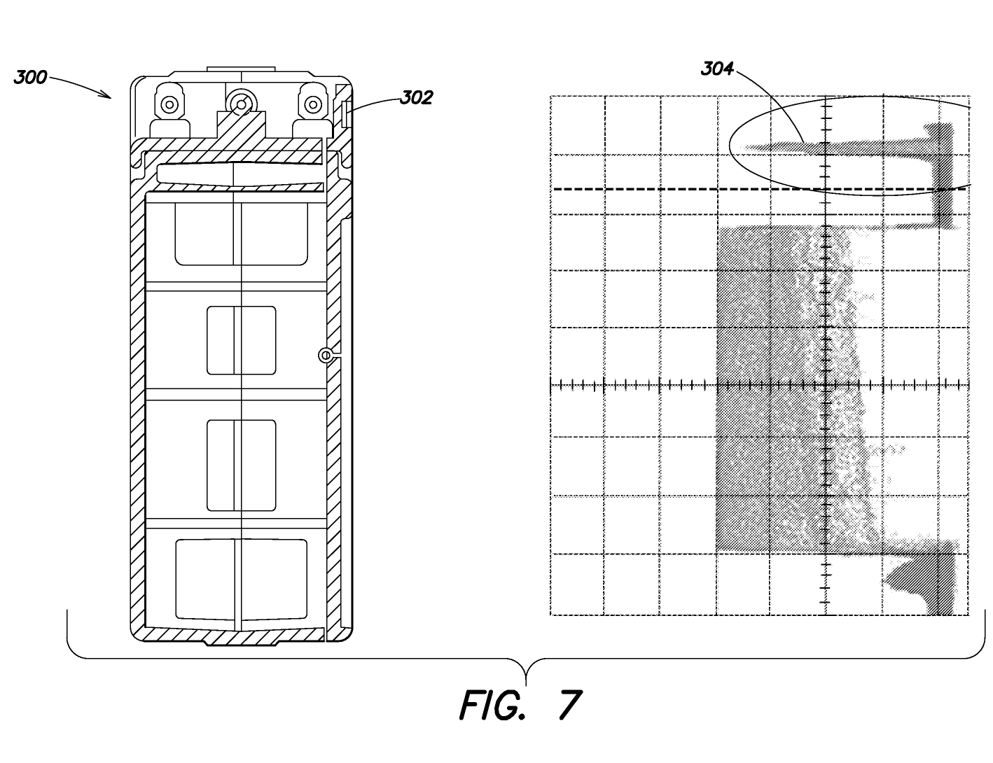

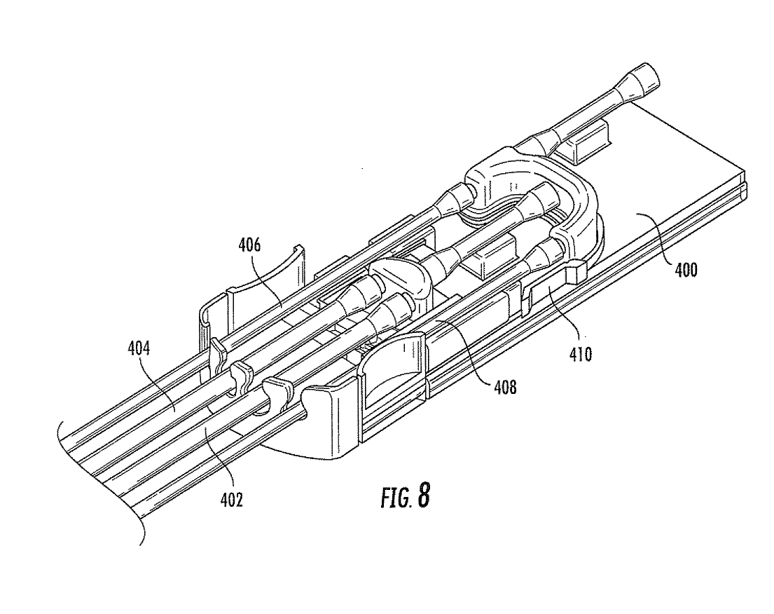

Referring again to FIG. 5 as shown, the spike 104 is sufficiently distinguished to be reliably detected by a detector of an ophthalmic surgical console. In other embodiments, one or more surfaces of an ophthalmic surgical cassette may be treated to minimize the refraction and/or reflection of light from remaining surfaces of an ophthalmic surgical cassette. Specifically as illustrated in FIG. 7, an anterior ophthalmic surgical cassette 300, includes multiple treated surfaces as is well known in the art, to minimize refraction and/or reflection of light (for example, surfaces surrounding feature 302 could be painted black). Accordingly, a spike 304 from an identification feature 302, included in the ophthalmic surgical cassette 300, is easily detectable by an ophthalmic surgical console. It should be appreciated that while the surface treatment of areas of an ophthalmic surgical cassette and manifold surrounding feature 302 may increase the accuracy of detecting an identification feature, positioning an identification feature on a manifold base of the ophthalmic surgical cassette provides sufficient differentiation of a spike, as shown in FIG. 5. Alternatively for example, an identification feature included in a container may require surface treatment to sufficiently differentiate a spike refracted from the identification feature from one or more other refracted/reflected light waveforms in the level sensing portion of a cassette. Surface treatments reduce/eliminate refraction and/or reflection of a container and/or other components of an ophthalmic surgical cassette providing an additional step in manufacturing, thereby adding cost.

Referring again to FIG. 1, the ophthalmic surgical cassette 10 includes a fluid level indicator 38. The fluid level indicator 38 includes a prism, which refracts light similar to the illustrated refraction of FIG. 4. Light is refracted in a first manner above a top surface of aspirant fluid and/or tissue in the ophthalmic surgical cassette, and in a second manner below the top surface of aspirant fluid and/or tissue in the ophthalmic surgical cassette. Accordingly, depending on a detector included in an ophthalmic surgical console, the fluid level indicator 38 may simply indicate when the aspirant fluid and/or tissue has reached a pre-determined point, e.g., full, etc., or when aspirant fluid and/or tissue reaches various other levels within an ophthalmic surgical cassette. Further as shown in FIG. 1, the fluid level indicator 38 is adjacent to and/or inline with the identification feature 22. In this manner, a single detector, e.g., an array of photo-detectors, etc., may be employed to detect the identification feature 22, and a level of aspirant fluid and/or tissue within the ophthalmic surgical cassette 10. It should be appreciated that an identification feature may be disposed differently on a manifold base of an ophthalmic surgical cassette in other embodiments of the present disclosure.

According to other embodiments of the present disclosure, a manifold base may be configured for more than one type of ophthalmic surgical procedure. FIG. 8 illustrates a manifold base 400 to be removeably coupled to a rigid-walled container for one or more posterior ophthalmic surgical procedures. The manifold base 400 includes an inlet irrigation tube 402 for coupling an irrigation source, and outlet irrigation tube 404 for providing irrigation to a surgical site, and an aspiration tube 406 for coupling an ophthalmic surgical handpiece. The manifold base 400 also includes an auxiliary aspiration tube 408 for coupling a second ophthalmic surgical handpiece or other ophthalmic surgical equipment configured to provide aspiration. In various embodiments, an auxiliary aspiration tube 408 may be included so that a surgeon does not have to change aspiration tubes between multiple ophthalmic surgical handpieces during an ophthalmic surgical procedure. Further, the manifold base 400 includes an identification feature 410, similar to the refraction element described above. The identification feature 410 provides an indication of a cassette type for a posterior ophthalmic surgical procedure. Because the cassette type is posterior, an ophthalmic surgical cassette, including the manifold base 400 installed in an ophthalmic surgery pump system, may cause surgical functions for anterior procedures to be disabled.

Alternatively if a cassette type is combined, an ophthalmic surgery pump system may not disable any surgical routines for anterior and/or posterior ophthalmic surgical procedures. In at least one embodiment, an ophthalmic surgical cassette with a cassette type of posterior or combined may not disable surgical functions/routines associated with the other of posterior and combined.

Although several aspects of the present disclosure have been described above with reference to ophthalmic surgical cassettes, it should be understood that various aspects of the present disclosure are not limited to ophthalmic surgical cassettes, and can be applied to a variety of other ophthalmic surgical systems, devices, and methods.

By implementing any or all of the teachings described above, a number of benefits and advantages can be attained, including improved reliability, reduced down time, elimination or reduction of redundant components or systems, avoiding unnecessary or premature replacement of components or systems, and a reduction in overall system and operating costs.

We claim:

1. An ophthalmic surgical cassette for use with an ophthalmic surgical console, the ophthalmic surgical cassette comprising:
a rigid-walled container having an interior volume for collecting aspirant fluid and/or tissue;
a manifold base, including an irrigation tube for coupling an irrigation source, an aspiration tube for coupling an ophthalmic surgical handpiece, and an identification feature for indicating a cassette type to an ophthalmic surgical console, the manifold base being removeably attached to the rigid-walled container; and
whereby the ophthalmic surgical console detects the identification feature to enable, based on the cassette type, at least one surgical function.

2. The ophthalmic surgical cassette of claim 1, wherein the identification feature is a refraction element.

3. The ophthalmic surgical cassette of claim 1, wherein the rigid-walled container includes a fluid level indicator for indicating a level of aspirant fluid and/or tissue in the rigid-walled container.

4. The ophthalmic surgical cassette of claim 3, wherein the identification feature is disposed inline with the fluid level indicator, such that a linear detector is capable of detecting the identification feature and the fluid level indicator.

5. The ophthalmic surgical cassette of claim 1, wherein the manifold base includes an auxiliary aspiration tube.

6. An ophthalmic surgical cassette for use with an ophthalmic surgical console, the ophthalmic surgical cassette comprising:
a rigid-walled container having an interior volume for collecting aspirant fluid and/or tissue and a fluid level indicator for refracting light generated by an ophthalmic surgical console to indicate a level of aspirant fluid and/or tissue within the rigid-walled container;
a manifold base removeably attached to the rigid-walled container, the manifold base, including an irrigation tube for coupling an irrigation source, an aspiration tube for coupling an ophthalmic surgical handpiece, and a refraction element for indicating a cassette type to the ophthalmic surgical console, the refraction element being disposed inline with the fluid level indicator; and
whereby the cassette type and the level of aspirant fluid and/or tissue are detectable by a photo-detector included in the ophthalmic surgical console.

7. An ophthalmic surgery pump system for use during an ophthalmic surgery procedure, the ophthalmic surgery pump system comprising:
an ophthalmic surgical cassette including a rigid-walled container and a manifold base removeably attached to the rigid-walled container, the manifold base, including an irrigation tube, an aspiration tube, and an identification feature for indicating a cassette type; and
an ophthalmic surgical console, including a detector disposed adjacent to the ophthalmic surgical cassette for detecting the identification feature and a processor operably coupled to the detector for enabling, based on the cassette type, at least one surgical function.

8. The ophthalmic surgery pump system of claim 7, wherein the identification feature includes a refraction element.

9. The ophthalmic surgery pump system of claim 8, wherein the identification feature includes a prism.

10. The ophthalmic surgery pump system of claim 8, wherein the detector includes a linear CCD array.

11. The ophthalmic surgery pump system of claim 10, wherein the ophthalmic surgical cassette includes a fluid level indicator disposed inline with the identification feature.

12. The ophthalmic surgery pump system of claim 11, wherein the linear CCD array extends adjacent to the identification feature and the fluid level indicator, such that the identification feature and the fluid level indicator are detectable by the linear CCD array.

13. The ophthalmic surgery pump system of claim 8, wherein the processor is configured to disable at least one surgical function of the ophthalmic surgical console based on the cassette type.

14. The ophthalmic surgery pump system of claim 8, wherein the manifold base includes an auxiliary aspiration tube.
